(19) United States
(12) Patent Application Publication
(10) Pub. No.: US 2025/0352139 A1
(43) Pub. Date: Nov. 20, 2025

(54) SYSTEM AND METHOD FOR SENSOR MONITORING OF RNA THERAPIES

(71) Applicant: COGNIGENICS, INC., Stuart, FL (US)

(72) Inventors: John MEE, Stuart, FL (US); Dean RADIN, Stuart, FL (US); Fabio MACCIARDI, Stuart, FL (US)

(21) Appl. No.: 19/208,306

(22) Filed: May 14, 2025

Related U.S. Application Data
(60) Provisional application No. 63/647,461, filed on May 14, 2024.

(51) Int. Cl. A61B 5/00; A61B 5/024; A61B 5/16

(52) U.S. Cl. CPC A61B 5/4839; A61B 5/0002; A61B 5/02405; A61B 5/165; A61B 5/6826

(57) ABSTRACT

A method of determining a dose of a therapeutic composition to a patient is provided. The patient is wearing a wearable device configured to detect at least one physiological parameter of the patient. The method includes detecting the at least one physiological parameter of the patient. The method further includes transmitting data associated with the at least one physiological parameter of the patient to a medical professional. The method also includes determining the dose of the therapeutic composition based on the data associated with the at least one physiological parameter of the patient.

### CROSS REFERENCE TO RELATED APPLICATION

This application claims the benefit of U.S. Provisional Patent Application No. 63/647,461, filed 14 May 2024, the disclosure of which is incorporated herein, in its entirety, by this reference.

### TECHNICAL FIELD

The present disclosure relates generally to systems and methods for monitoring RNA therapies.

### BACKGROUND OF CERTAIN ASPECTS OF THE DISCLOSURE

RNA therapies may be used to treat certain psychiatric conditions that are treated based on the subjective reporting of a patient to a medical professional and are treated with medications that can take an extended amount of time to take effect. For example, some selective serotonin reuptake inhibitors (SSRIs) may be prescribed for certain psychiatric conditions but may take weeks to take effect. Additionally, some patients may not be in as in tune with their emotions as other patients. For example, some patients may not realize that their stress levels are rising but their physiological indicia may give medical professionals an indication that the patient's stress levels are rising before the patient has this insight. The treatment of the patient may be improved by a system that provides a medical professional with information about the patient's physiological indicia to guide the medical professional's analysis and future treatment plans.

As a result, improved methods of providing medical professionals with information about the patient's physiological indicia are needed.

### BRIEF SUMMARY OF SOME ASPECTS OF THE DISCLOSURE

One aspect of the present disclosure relates to methods of determining a dose of a therapeutic composition to be administered to a patient. The patient is wearing a wearable device configured to detect at least one physiological parameter of the patient. The method includes detecting the at least one physiological parameter of the patient. The method further includes transmitting data associated with the at least one physiological parameter of the patient to a medical professional. The method also includes determining the dose of the therapeutic composition based on the data associated with the at least one physiological parameter of the patient.

There are other novel aspects and features of this disclosure. They will become apparent as this specification proceeds. Accordingly, this brief summary is provided to introduce a selection of concepts in a simplified form that are further described below in the detailed description. The summary and the background are not intended to identify key concepts or essential aspects of the disclosed subject matter, nor should they be used to constrict or limit the scope of the claims. For example, the scope of the claims should not be limited based on whether the recited subject matter includes any or all aspects noted in the summary and/or addresses any of the issues noted in the background.

### BRIEF DESCRIPTION OF THE DRAWINGS

A further understanding of the nature and advantages of the embodiments may be realized by reference to the following drawings. In the appended figures, similar components or features may have the same reference label.

FIG. 1 illustrates a flow diagram of a method of determining a dose of a therapeutic composition to a patient in accordance with aspects of the present disclosure.

FIG. 2 illustrates durations of action for different RNA therapies in accordance with aspects of the present disclosure.

FIG. 3 illustrates an example of a system including a device in accordance with aspects of the present disclosure.

FIG. 4 illustrates an example of a system including a device in accordance with aspects of the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The systems and methods disclosed herein relate to, among other things, detecting, storing, retrieving, and analyzing patient biometric information to guide the administration of gene therapies for improving the mental health and cognition of a subject such as a human patient. Specifically, the systems and methods described herein relate to measuring one or more physiological indicia from a patient and treating the patient on the basis of determinations made using the one or more physiological indicia. More specifically, in some embodiments, the systems and methods described herein relate to receiving one or more physiological indicia from a patient using a wearable device and treating the patient using the one or more physiological indicia where the treatment involves treating a psychiatric condition with an RNA therapy.

In the illustrated embodiments, the patient wears a wearable device configured to detect one or more physiological indicia. The wearable device sends the one or more physiological indicia to a computing system that may include at least one artificial intelligence system that analyzes the one or more physiological indicia. The system reports the data, a summary of the data, and a preliminary analysis of the data to a medical professional. The medical professional then completes the analysis based at least partly on the analysis provided by the artificial intelligence system and may revise a treatment plan for the patient based on the analysis.

More specifically, in the illustrated embodiment, certain psychiatric conditions are treated based on the subjective reporting of the patient to the medical professional. In some instances, at least some psychiatric conditions are treated with medications that can take an extended amount of time to take effect. For example, some selective serotonin reuptake inhibitors (SSRIs) may be prescribed for certain psychiatric conditions but may take weeks to take effect. Additionally, some patients may not be in as in tune with their emotions as other patients. For example, some patients may not realize that their stress levels are rising but their physiological indicia may give medical professionals an indication that the patient's stress levels are rising before the patient has this insight. The wearable device enables the medical professional to monitor certain physiological indicia that may provide the medical professional with an insight into how the patient is feeling.

For example, the wearable device may monitor a physiological parameter such as the patient's heart rate variability (HRV) to determine the patient's stress levels. HRV tends to correlate well for overall stress of the patient. As such, if the patient sees a psychiatrist and the psychiatrist prescribes a medication that takes weeks to take effect, like an SSRI, the psychiatrist may then continually adjust the medication based on the subjective report that the patient provides. In contrast, the systems and methods described herein automatically and continually provide physiological data in order to give the medical professional an insight into the specific patient's level of stress. If the medical professional receives data that indicates that the patient's stress level is rising, whether the patient subjectively knows it or not, the medical professional can then message the patient that it is time for a checkup and a medication adjustment.

Accordingly, the systems and methods described herein provide medical professionals with automatic and continuous data about a patient, AI generated analysis of the data, the ability to analyze the patient's data between visits, and the ability to proactively make changes to a treatment plan based on the AI generated analysis and the patient's data. As such, the systems and methods described herein enable the medical professional to improve the quality of care provided to the patient.

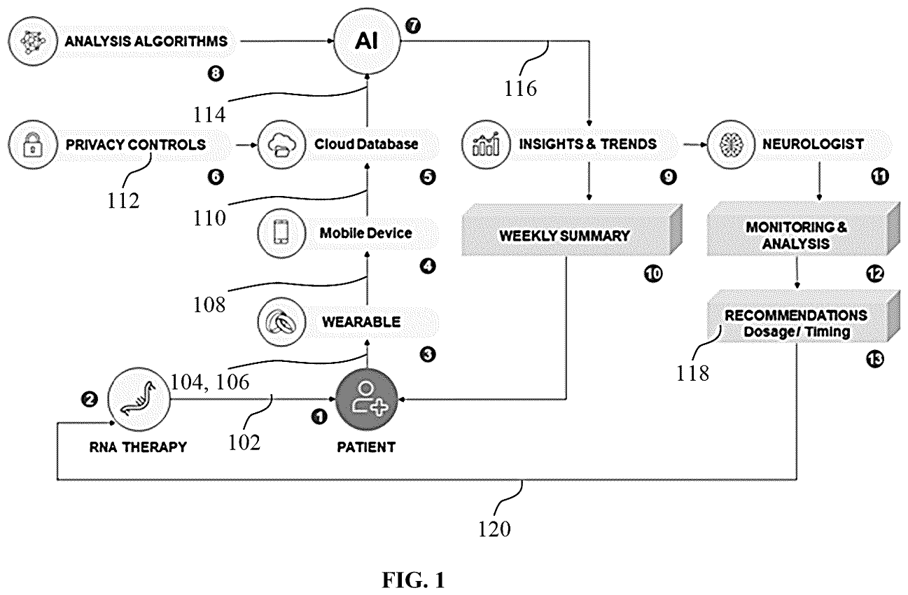

FIG. 1 illustrates a flow diagram of a method 100 of determining a dose of a therapeutic composition to a patient. In general, the method 100 includes determining the dosage of a gene therapy to improve the mental health and cognition of a patient. Additionally, method 100 provides patients with scientific feedback data which reinforces their belief in their capacity to change. As such, the method 100 described herein enables both patients and medical professionals to determine the appropriate dosage of a therapeutic medication to provide patients and medical professionals with feedback on the current dosage improve the patient's and medical professional's understanding of the patient's current state and to reinforce the patient's belief in their capacity to change.

Method 100 begins by administering 102 at least one dose of a therapeutic composition to a patient. In the illustrated embodiment, the therapeutic composition includes a ribonucleic acid (RNA) therapy. For example, the RNA therapies may include siRNA therapies, shRNA therapies, cRNA therapies, and/or any other RNA therapy. In alternative embodiments, the therapeutic composition may include any medication, supplement, treatment, therapy, and/or medical intervention of any sort than enables the systems and methods described herein to operate as described herein. The therapeutic composition may be administered by a medical practitioner, another non-medical practitioner, and/or by the patient.

In the illustrated embodiment, the therapeutic composition includes an RNA therapy which modifies neuron electrodynamic excitability in the brain's limbic region to treat common mental health issues such as stress, anxiety, depression, PTSD and ADD, as well as personality disorders. More specifically, as described herein, the RNA therapy enables a region of the patient's brain to be activated such that a drug delivered intranasally can target the specific activated region of the brain to treat a specific mental health condition.

Common mental health issues such as stress, anxiety, depression and inattention are all experimentally correlated with overactive neurons in specific areas of the limbic system. Neuron activity levels in these areas can be normalized through gene therapies which alter a neuron's structure to lower its electrical excitability. The RNA therapy allows for delivery of neuron-editing biologics to targeted regions and connectomes of the brain to achieve specific neurological treatment or cognitive enhancement goals.

Generally, the RNA therapy generally includes the steps of (1) assessment of the patient/subject to determine issues and establish goals; (2) identification of brain regions to be treated; (3) pre-treatment/cognitive therapy of the patient; (4) prescription and administration of selected biologics to the patient; and (5) post-treatment/cognitive therapy.

In specific embodiments, the RNA therapy enables intranasal delivery of neuron-editing biologics to the olfactory epithelium via a swab or nasal inhaler. The olfactory bulb pathways transport the biologics directly to the limbic lobe and the neuron-editing biologics target the limbic system to treat mental health conditions such as, but not limited to, inattention, stress, anxiety and depression. In some instances, the method 100 may be used to attenuate hyperactive or hypoactive neurons in the limbic system which are responsible for inattention (ventral posterior cingulate cortex (PCC)), stress (amygdala, hippocampus), anxiety (amygdala), and depression (amygdala, hippocampus).

Advantages of the RNA therapy include (1) limiting biologics delivery to the region of interest to minimize dosing requirements and avoid affecting other areas of the brain; (2) avoid damage to biologic agents by avoiding transit which would expose them to digestive enzymes or immune system response by delivering them intranasally; (3) alleviating hippocampal and amygdaloid hyperactivity or hypoactivity which may also improve memory and could potentially halt the progression of dementia and extend longevity; and (4) in addition to one-time DNA edits, some applications may involve multiple iterations of DNA edits. For example, the multiple iterations of DNA edits may include layered DNA edits, where the edit is divided into multiple smaller doses, or temporary RNA edits, which are repeated over time. Adeno-associated viral vectors have been used for editing neurons, but after one systemically-delivered application, the body develops an immune response to the adeno-associated viral vectors, preventing further uses. However, using viral vectors for multiple iterations is feasible with intranasal delivery because it transports gene editing payloads directly into the brain without activating an immune system response.

Once inside the limbic lobe, the RNA therapy includes three further methods to direct neuron-editing biologics to a target region of the patient's brain. First, actively-directed hemodynamic vectoring can transport the biologics to specific areas of the patient's brain which are energized by brain region activators. Second, passive hemodynamic vectoring will naturally transport the biologics to the most active neurons of the patient's brain. Finally, ligand vectors including targeting peptide nanoparticles designed to direct editing biologics to neuron cell types may be used to direct them to specific brain regions of the patient's brain. Hemodynamic and ligand vectoring are complementary therapies. Ligand vectors may be pre-programmed to statically treat specific topographical areas of brainwave activity imbalance identified by a neuroimaging assessment. Hemodynamic vectors can dynamically treat in real-time systemic electrophysiological activity imbalances stimulated, for example, by psychotherapy, virtual reality, and/or perceptual events, as well as statically targeting specific regions of the brain. However, ligand vectors can transport biologics only to neurons which have the receptor types of interest by sensing proteins which are unique to each receptor type, limiting the types of neurons that can be treated with ligand vectors. Method 100 enables biologics to edit any type of neuron by activating the neuron prior to intranasal delivery of the biologic, enabling the biologic to edit the types of neurons that ligand vectoring cannot edit.

Thus, the RNA therapy may include a specific-purpose neurological condition reliever which works on individual conditions (e.g., inattention, stress, anxiety, depression) by genetically altering neuron structure in order to modify neuron electrodynamics. Method 100 relieves the conditions by intranasal delivery of neuron-editing biologics to the limbic system in combination with (1) actively-directed hemodynamic vectoring, (2) passive hemodynamic vectoring, and/or (3) ligand vectors.

However, the RNA therapies described herein may have a limited duration of action and may have a long onset period. That is, RNA therapies for mental health are expected to have limited durations of action in the range of 2-6 months, depending on dosage and patient predisposition, with sustainable results achieved via repeat doses which are administered when effects taper off. Because EEG brain scans do not reliably detect anxiety, the only current method for determining the patient's anxiety level is their own subjective reporting. Because the dose has a 5 week onset period, there may be valleys in overall efficacy if the repeat dose is delayed until the patient experiences a noticeable tapering off of effects.

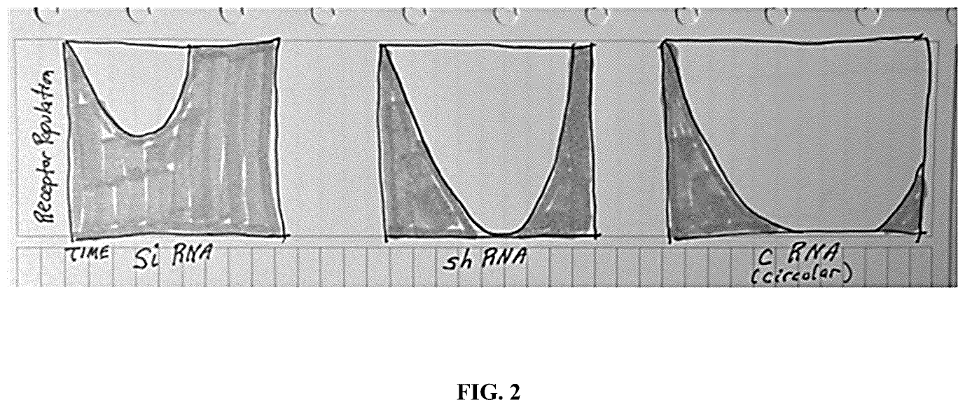

For example, FIG. 2 illustrates durations of action for different RNA therapies. As shown in FIG. 2, siRNA is expected to have a duration of action of several days, shRNA is expected to have a duration of action of several months, and cRNA is expected to have a duration of action of six months. That is, RNA therapies for mental health have different active lives in neurons depending on the type of RNA interference molecule utilized. While the compound is active in the neuron, it blocks transcription of its target gene into information for building the cell proteins to replace receptors which naturally break down. Receptor population declines as old receptors are recycled and not replaced with new ones. Once the RNA interference compound itself breaks down, the cell will rebuild its receptors. Receptor population will follow the parabolic patterns illustrated in FIG. 2.

Thus, the RNA therapies described herein will periodically need to be readministered and the period of re-administration may very based on the patient's physiology and other environmental factors.

The method 100 also includes setting up and attaching 104 a wearable device including at least one sensor to the patient. In some embodiments, the wearable device includes a watch or a ring including a sensor configured to detect at least one physiological parameter of the patient. In alternative embodiments, the wearable device may include devices that remain attached to the patient for an extended period of time. For example, the wearable device may include devices like glucose monitors, insulin pumps, and/or other semipermanent or permanently attached devices that include a sensor configured to detect at least one physiological parameter of the patient.

In the illustrated embodiment, the wearable device generally includes smart watches and/or smart rings configured to detect at least one physiological parameter of the patient. The at least one physiological parameter of the patient may include at least one of a temperature, a pulse, a locomotion, or a heart rate variability of the patient. Additionally, the at least one sensor may include any type of biometric sensor that enables the systems and methods described herein to operate as described herein. For example, in some embodiments, the at least one sensor may include a heart rate sensor of any type (electrical, optical, and/or any others), a thermometer, an accelerometer, an altimeter, and/or any other type of sensor.

The method 100 further includes measuring 106 the at least one physiological parameter of the patient using the at least one sensor. In the illustrated embodiment, the wearable device is worn by the patient and the at least one sensor periodically or continuously measures the at least one physiological parameter of the patient. In alternative embodiments, the wearable device measures the at least one physiological parameter of the patient only when directed to do so by the patient.

The method 100 also includes transmitting 108 data associated with the at least one physiological parameter of the patient to a computing device. In some embodiments, the computing device includes any combination of, for example, mobile devices, smart phones, personal computing devices, computers, laptops, desktops, servers, media content set top boxes, or any combination thereof. However, the computing device may be any device that enables the systems and methods described herein to operate as described herein.

In some embodiments, transmitting 108 data associated with the at least one physiological parameter of the patient to the computing device includes transmitting data associated with the at least one physiological parameter of the patient from the wearable device to the computing device using a wireless communications system such as any past, present, or current version of Bluetooth. The wireless communications system may also include code division multiple access (CDMA), time division multiple access (TDMA), frequency division multiple access (FDMA), orthogonal frequency division multiple access (OFDMA), single carrier frequency division multiple access (SC-FDMA), and other systems. The terms “system” and “network” are often used interchangeably. A code division multiple access (CDMA) system may implement a radio technology such as CDMA2000, Universal Terrestrial Radio Access (UTRA), etc. CDMA2000 covers IS-2000, IS-95, and IS-856 standards. IS-2000 Releases may be commonly referred to as CDMA2000 1×, 1×, etc. IS-856 (TIA-856) is commonly referred to as CDMA2000 1×EV-DO, High Rate Packet Data (HRPD), etc. UTRA includes Wideband CDMA (WCDMA) and other variants of CDMA. A time division multiple access (TDMA) system may implement a radio technology such as Global System for Mobile Communications (GSM). An orthogonal frequency division multiple access (OFDMA) system may implement a radio technology such as Ultra Mobile Broadband (UMB), Evolved UTRA (E-UTRA), IEEE 805.11 (Wi-Fi), IEEE 805.16 (WiMAX), IEEE 805.20, Flash-OFDM, etc.

The method 100 further includes transmitting 110 data associated with the at least one physiological parameter of the patient to a server. In some embodiments, the server may include a data server, a cloud server, proxy server, mail server, web server, application server, database server, communications server, file server, home server, mobile server, name server, or any combination thereof. However, the server may be any computing device that enables the systems and methods described herein to operate as described herein.

In some embodiments, transmitting 110 data associated with the at least one physiological parameter of the patient to the server includes transmitting data associated with the at least one physiological parameter of the patient from the computing device to the server via a network. Examples of the network may include any combination of cloud networks, local area networks (LAN), wide area networks (WAN), virtual private networks (VPN), wireless networks (using 805.11, for example), cellular networks (using 3G, LTE, or 5G, for example), etc. In some configurations, the network may include the Internet. In alternative embodiments, the wearable device may communicate directly with the server via the networks described herein.

The method 100 also optionally includes protecting 112 data associated with the at least one physiological parameter of the patient with privacy controls. The privacy controls may include protocols, programs, applications, and/or algorithms that are compliant with any of the following security standards and laws: ISO/IEC 17789, ISO/IEC 19944-1, ISO/IEC Technical Specification 23167, ISO/IEC 27018, Payment Card Industry Data Security Standard, Health Insurance Portability and Accountability Act (HIPAA), General Data Protection Regulation (GDPR), System and Organization Controls (SOC) CC2.0, SOC CC5.0, SOC CC6.0, SOC CC7.0, SOC CC8.0, and/or any other privacy and security standard, law, and/or protocol.

The method 100 further includes analyzing 114 data associated with the at least one physiological parameter of the patient with artificial intelligence and analysis algorithms. An artificial intelligence and analysis algorithm platform includes a set of datastores that store a governance library that defines a set of governance standards that include at least one set of security standards, legal standards, ethical standards, regulatory standards, quality standards, or engineering standards that are applied to decisions made by one or more respective intelligence services. The artificial intelligence and analysis algorithm platform includes a set of one or more processors that execute a set of computer-readable instructions. The set of one or more processors collectively execute a governance-enabling intelligence layer that receives and responds to intelligence requests received from respective intelligence service clients. The intelligence layer includes a set of artificial intelligence services that includes at least one of a machine learning service, a rules-based intelligence service, a digital twin service, an automation service, or a machine service. The intelligence layer includes an intelligence layer controller that coordinates performance of respective intelligence services on behalf of the respective intelligence service clients and performance of a set of analyses corresponding to the respective intelligence services based in part on the set of governance standards. The intelligence layer returns decisions determined collectively by the artificial intelligence service in response to the intelligence requests, such that the decisions are determined based on a set of intelligence service data sources and the set of analyses. More specifically, in the illustrated embodiment, the artificial intelligence and analysis algorithm platform includes an intelligence layer controller that coordinates performance of respective intelligence services that are configured to analyze at least one physiological parameter of the patient, make a recommendation about treatment based on the analysis, and provide the medical professional with the recommendation and the data the recommendation was based on.

The method 100 also includes sending 116 insights and analysis based on the analysis performed by the artificial intelligence and analysis algorithms. More specifically, in some embodiments, the server may send a periodic summary of the analysis performed by the artificial intelligence and analysis algorithms to the patient. In some cases, the periodic summary may be sent daily, weekly, and/or monthly. In other cases, the patient and/or medical professional may set the frequency of the periodic summary sent to the patient. Additionally, the insights and analysis based on the analysis performed by the artificial intelligence and analysis algorithms may also be sent to the medical professional on a periodic basis, an as needed basis, and/or on demand basis. For example, the server may only send the insights and analysis based on the analysis performed by the artificial intelligence and analysis algorithms to the medical professional only if a predetermined parameter is below (or above) a certain predetermined threshold. For example, if the HRV of the patient is too low, that may indicate that the patient is experiencing higher levels of stress and the system may alert the medical professional that intervention may be required. In other embodiments, the server may be programmed only to provide the insights and analysis based on the analysis performed by the artificial intelligence and analysis algorithms on demand or on a periodic basis.

The method 100 further includes monitoring, analysis, and treatment 118 by the medical professional based on the insights and analysis based on the analysis performed by the artificial intelligence and analysis algorithms. Specifically, the medical professional may analyze and evaluate the insights and analysis based on the analysis performed by the artificial intelligence and analysis algorithms and determine the appropriate path forward. This may include changing the patient's treatment plan including changing the dosing of the therapeutic composition.

Method 100 may then continuously iterate 120, and the medical professional may then continue to refine the patient's treatment plan based on the analysis performed by the artificial intelligence and analysis algorithms.

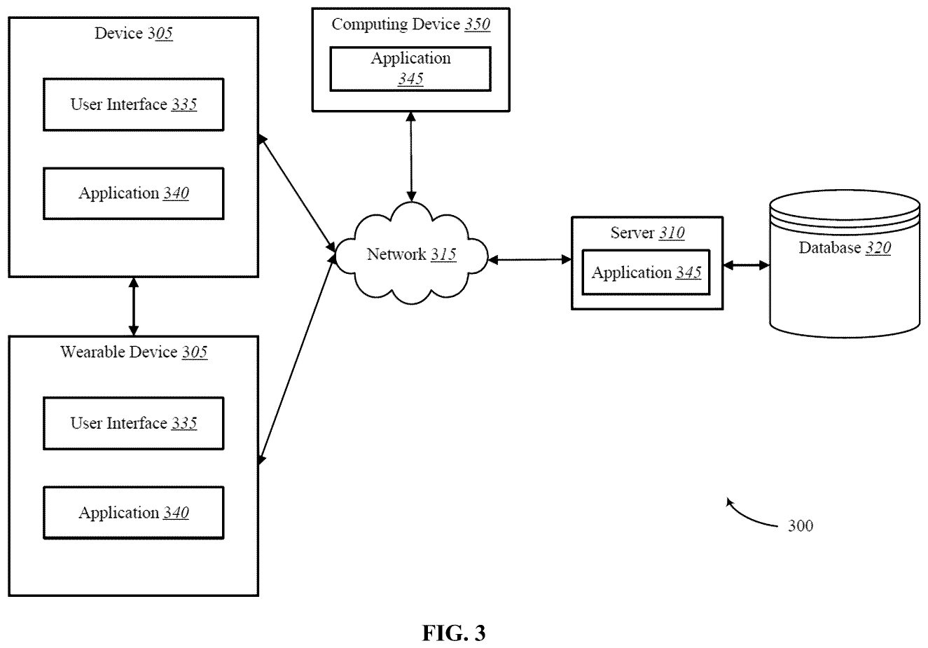

FIG. 3 illustrates a block diagram illustrating one example of a system 300 in which the present systems and methods may be implemented. In some examples, the systems and methods described herein may be performed on a device (e.g., device 305). As depicted, the system 300 may include a device 305, a server 310, a network 315, a database 320, and a computing device 310, and that allows the device 305, the server 310, and the database 320 to communicate with one another.

Examples of the device 305 may include any combination of, for example, mobile devices, smart phones, personal computing devices, computers, laptops, desktops, servers, media content set top boxes, or any combination thereof. However, the device 305 may be any device that enables the systems and methods described herein to operate as described herein.

Examples of computing device 310 may include at least one of one or more client machines, one or more mobile computing devices, one or more laptops, one or more desktops, one or more servers, one or more media set top boxes, or any combination thereof. However, the computing device 310 may be any computing device that enables the systems and methods described herein to operate as described herein.

Examples of server 310 may include, for example, a data server, a cloud server, proxy server, mail server, web server, application server, database server, communications server, file server, home server, mobile server, name server, or any combination thereof. However, the server 310 may be any computing device that enables the systems and methods described herein to operate as described herein.

Although database 320 is depicted as connecting to device 305 via network 315, in some examples, device 305 may connect directly to database 320. In some examples, device 305 may connect or attach to at least one of database 320 or server 310 via a wired or wireless connection, or both. In some examples, device 305 may attach to any combination of a port, socket, and slot of a separate computing device or server 310.

In some configurations, the device 305 may include a user interface 335 and an application 340. Although the components of the device 305 are depicted as being internal to the device 305, it is understood that one or more of the components may be external to the device 305 and connect to the device 305 through wired or wireless connections, or both. Examples of the application 340 may include a web browser, a software application, a desktop application, a mobile application, etc. In some examples, the application 340 may be installed on a computing device in order to allow a user to interface with a function of the device 305, the server 310, and the computing device 310.

Although the device 305 is illustrated with an exemplary single application 340, in some examples the application 340 may represent two or more different applications installed on, running on, or associated with the device 305. In some examples, the application 340 may include one or more software widgets. In some cases, the application 340 may include source code to operate one or more of the systems, system components, and/or methods described herein.

In some examples, the device 305 may communicate with the server 310 via the network 315. Examples of the network 315 may include any combination of cloud networks, local area networks (LAN), wide area networks (WAN), virtual private networks (VPN), wireless networks (using 805.11, for example), cellular networks (using 3G, LTE, or 5G, for example), etc. In some configurations, the network 315 may include the Internet. For example, the device 305 may include the application 340 that allows the device 305 to interface with a separate device via an application 345 being located on another device such as a separate computing device, server 310, database 320, or any combination thereof.

In some examples, at least one of the devices 305, the database 320, and the server 310 may include an application 345 where at least a portion of the functions of the application 340 are performed separately or concurrently on the device 305, the database 320, and/or the server 310. In some examples, a user may access the functions of the device 305 (directly or through the device 305 via the application 345) from the database 320 or the server 310. In some examples, the database 320 includes a mobile application that interfaces with one or more functions of the device 305 and/or the server 310.

In some examples, the server 310 may be coupled to the database 320. The database 320 may be internal or external to the server 310. In one example, the device 305 may be coupled to the database 320. In some examples, the database 320 may be internally or externally connected directly to the device 305. Additionally or alternatively, the database 320 may be internally or externally connected directly to the computing device 310 or one or more network devices such as a gateway, switch, router, intrusion detection system, etc. The database 320 may include the application 345. In some examples, device 305 may access or operate aspects of the application 345 from the database 320 over the network 315 via the server 310. The database 320 may include script code, hypertext markup language code, procedural computer programming code, compiled computer program code, object code, uncompiled computer program code, object-oriented program code, class-based programming code, cascading style sheets code, or any combination thereof.

In one example, the device 305 may be coupled to the database 320. In some examples, the database 320 may be internally or externally connected directly to the device 305. Additionally or alternatively, the database 320 may be internally or externally connected directly to one or more network devices such as a gateway, switch, router, intrusion detection system, etc.

The application 345 may enable a variety of features and functionality related to the systems and methods described herein. In some examples, the application 345 may be configured to perform the systems and methods described herein in conjunction with the user interface 335 and the application 340. The user interface 335 may enable a user to interact with, control, or program one or more functions of the application 345.

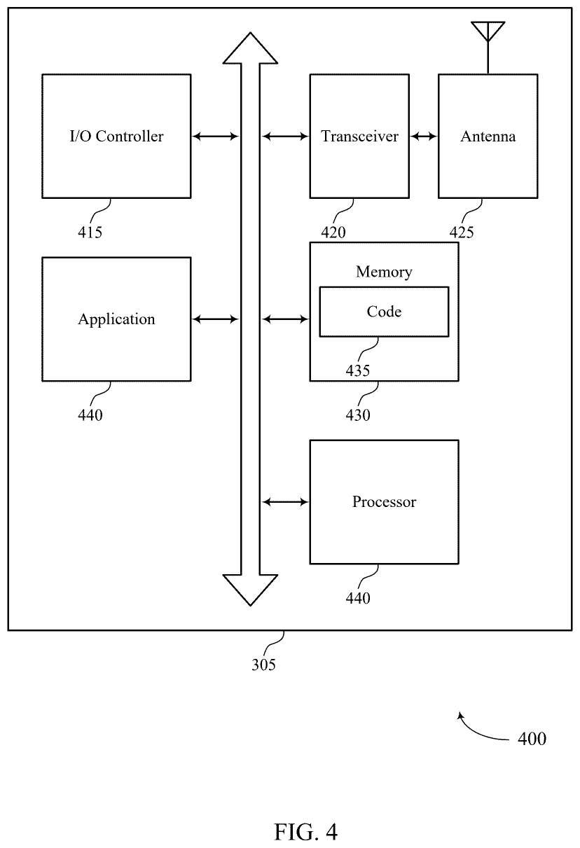

FIG. 4 shows a diagram of a system 400 including the device 405 that performs the systems and methods described herein. The device 405 may be an example of or include the components of device 305 or devices as described herein. The device 405 may include components for bi-directional voice and data communications including components for transmitting and receiving communications, including the application 340, an I/O controller 415, a transceiver 420, an antenna 425, memory 430, and a processor 440. These components may be in electronic communication via one or more buses.

The application 340 may provide any combination of the operations and functions described herein related to the systems and the methods described herein.

The I/O controller 415 may manage input and output signals for the device 405. The I/O controller 415 may also manage peripherals not integrated into the device 405. In some cases, the I/O controller 415 may represent a physical connection or port to an external peripheral. In some cases, the I/O controller 415 may utilize an operating system such as iOS®, ANDROID®, MS-DOS®, MS-WINDOWS®, OS/2®, UNIX®, LINUX®, or another known operating system. In other cases, the I/O controller 415 may represent or interact with a modem, a keyboard, a mouse, a touchscreen, or a similar device. In some cases, the I/O controller 415 may be implemented as part of a processor. In some cases, a user may interact with the device 405 via the I/O controller 415 or via hardware components controlled by the I/O controller 415.

The transceiver 420 may communicate bi-directionally, via one or more antennas, wired, or wireless links as described herein. For example, the transceiver 420 may represent a wireless transceiver and may communicate bi-directionally with another wireless transceiver. The transceiver 420 may also include a modem to modulate the packets and provide the modulated packets to the antennas for transmission, and to demodulate packets received from the antennas.

In some cases, the wireless device may include a single antenna 425. However, in some cases the device may have more than one antenna 425, which may be capable of concurrently transmitting or receiving multiple wireless transmissions.

The memory 430 may include RAM and ROM. The memory 430 may store computer-readable, computer-executable code 435 including instructions that, when executed, cause the processor to perform various functions described herein. In some cases, the memory 430 may contain, among other things, a BIOS which may control basic hardware or software operation such as the interaction with peripheral components or devices.

The processor 440 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 440 may be configured to operate a memory array using a memory controller. In other cases, a memory controller may be integrated into the processor 440. The processor 440 may be configured to execute computer-readable instructions stored in a memory (e.g., the memory 430) to cause the device 405 to perform various functions (e.g., functions or tasks supporting menu related functions and other functions associated with the systems and methods disclosed herein).

The code 435 may include instructions to implement aspects of the present disclosure, including instructions to support dynamic accessibility compliance of a website. The code 435 may be stored in a non-transitory computer-readable medium such as system memory or other type of memory. In some cases, the code 435 may not be directly executable by the processor 440 but may cause a computer (e.g., when compiled and executed) to perform functions described herein.

It should be noted that the methods described herein describe possible implementations, and that the operations and the steps may be rearranged or otherwise modified and that other implementations are possible. Furthermore, aspects from two or more of the methods may be combined.

If wireless communications are used, the techniques described herein may be used for various wireless communications systems such as code division multiple access (CDMA), time division multiple access (TDMA), frequency division multiple access (FDMA), orthogonal frequency division multiple access (OFDMA), single carrier frequency division multiple access (SC-FDMA), and other systems. The terms “system” and “network” are often used interchangeably. A code division multiple access (CDMA) system may implement a radio technology such as CDMA2000, Universal Terrestrial Radio Access (UTRA), etc. CDMA2000 covers IS-2000, IS-95, and IS-856 standards. IS-2000 Releases may be commonly referred to as CDMA2000 1×, 1×, etc. IS-856 (TIA-856) is commonly referred to as CDMA2000 1×EV-DO, High Rate Packet Data (HRPD), etc. UTRA includes Wideband CDMA (WCDMA) and other variants of CDMA. A time division multiple access (TDMA) system may implement a radio technology such as Global System for Mobile Communications (GSM). An orthogonal frequency division multiple access (OFDMA) system may implement a radio technology such as Ultra Mobile Broadband (UMB), Evolved UTRA (E-UTRA), IEEE 805.11 (Wi-Fi), IEEE 805.16 (WiMAX), IEEE 805.20, Flash-OFDM, etc.

The wireless communications system or systems described herein may support synchronous or asynchronous operation. For synchronous operation, the stations may have similar frame timing, and transmissions from different stations may be approximately aligned in time. For asynchronous operation, the stations may have different frame timing, and transmissions from different stations may not be aligned in time. The techniques described herein may be used for either synchronous or asynchronous operations.

The downlink transmissions described herein may also be called forward link transmissions while the uplink transmissions may also be called reverse link transmissions. Each communication link described herein may include one or more carriers.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein may be implemented using software executed by a processor, hardware, firmware, hardwiring, or combinations of any of these. Features implementing functions may also be physically located at various positions, including being distributed such that portions of functions are implemented at different physical venues. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable read-only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

### Terminology and Interpretative Conventions

As used herein, neuron editing biologics are biopharmaceuticals, in some cases protein-based, or peptide-based biopharmaceuticals, which can modify a neuron's DNA or RNA. These may include gene knockout biologics, gene silencing biologics, and/or gene knock-in biologics.

Gene knockout biologics may include a catalytically-active gene-editing endonuclease(s) complexed with a synthetic guide RNA. Gene silencing biologics may include a catalytically-inactive gene-editing endonuclease(s) complexed with a synthetic guide RNA. Gene knock-in biologics may include a gene-editing endonuclease(s) complexed with a gene-expression inhibiting nucleotide and a synthetic guide RNA.

As used herein, transcriptional engineering biologics may include RNA knockout, RNA silencing, RNA knock-in, RNA translational interference, and Micro-RNA biogenesis suppression compounds/systems. RNA knockout compounds may include a catalytically-active RNA-editing ribonuclease complexed with a single guide RNA to alter RNA nucleotides to repress gene translation. RNA silencing compounds may include is a catalytically-inactive RNA-editing ribonuclease complexed with a single guide RNA to bind to RNA nucleotides to repress or enhance gene translation. RNA knock-in biologic may include a catalytically-inactive RNA-editing ribonuclease complexed with a single guide RNA and a deaminase enzyme to cause RNA nucleobase substitutions which result in translational interference. RNA translational interference compounds may include an RNA-editing ribonuclease complexed with an RNA-expression inhibiting nucleotide and a single guide RNA to alter RNA nucleotides to cause translational interference. Micro-RNA biogenesis suppression compounds may include a catalytically-active ribonuclease complexed with a single guide RNA to alter nucleotides in biogenesis processing sites for micro-RNA used in the translation of genes in order to reduce their expression.

As used herein, brain region activators include methods and techniques for focusing and concentrating the neuronal activity in a patient's brain into a specific, targeted area of the brain.

As used herein, transcranial pulsed ultrasound is a technique which uses low-power, low-frequency ultrasound to stimulate high neuron activity in the brain. It may be directed to any brain region and precisely focused to areas as small as several cubic millimeters.

As used herein, transcranial magnetic stimulation is a form of neurostimulation which uses a shallow magnetic field to induce electric current to flow in small targeted regions near the surface of the brain.

As used herein, perceptual isolation is the deliberate removal of stimuli from the senses of a subject/patient. Examples of perceptual isolation methods include sleep masks, white noise, soundproofing, and/or floatation tanks.

As used herein, neurofeedback is a type of biofeedback that measures brain waves to produce a signal that can be used as feedback to teach self-regulation of brain function. Patients can be trained to alter their brain activity to increase performance on certain tasks, which in turn changes the signal and increases cerebral blood flow to a specified region of the brain.

As used herein, virtual reality may be an immersive, interactive, computer-generated experience which occurs in a simulated environment including auditory and visual feedback.

As used herein, psychotherapy is a wide field encompassing the use of any one or more of hundreds of different methods and techniques to improve an individual's well-being, behavior and mental health, including cognitive therapy and other forms of therapies.

Any methods described in the claims or specification should not be interpreted to require the steps to be performed in a specific order unless stated otherwise. Also, the methods should be interpreted to provide support to perform the recited steps in any order unless stated otherwise.

Spatial or directional terms, such as “left,” “right,” “front,” “back,” and the like, relate to the subject matter as it is shown in the drawings. However, it is to be understood that the described subject matter may assume various alternative orientations and, accordingly, such terms are not to be considered as limiting.

Articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items.

The terms have, having, include, and including should be interpreted to be synonymous with the terms comprise and comprising. The use of these terms should also be understood as disclosing and providing support for narrower alternative embodiments where these terms are replaced by “consisting” or “consisting essentially of.”

Unless otherwise indicated, all numbers or expressions, such as those expressing dimensions, physical characteristics, and the like, used in the specification (other than the claims) are understood to be modified in all instances by the term “approximately.” At the very least, and not as an attempt to limit the application of the doctrine of equivalents to the claims, each numerical parameter recited in the specification or claims which is modified by the term “approximately” should be construed in light of the number of recited significant digits and by applying ordinary rounding techniques.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

All disclosed numerical values are to be understood as being variable from 0-100% in either direction and thus provide support for claims that recite such values or any and all ranges or subranges that can be formed by such values. For example, a stated numerical value of 8 should be understood to vary from 0 to 16 (100% in either direction) and provide support for claims that recite the range itself (e.g., 0 to 16), any subrange within the range (e.g., 2 to 12.5) or any individual value within that range (e.g., 15.2).

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained in this document should be considered a disclaimer or disavowal of claim scope.

The subject matter recited in the claims is not coextensive with and should not be interpreted to be coextensive with any embodiment, feature, or combination of features described or illustrated in this document. This is true even if only a single embodiment of the feature or combination of features is illustrated and described in this document.

### INCORPORATED BY REFERENCE

The following patents and applications are incorporated by reference:

U.S. Provisional Pat. App. No. 63/551,460 entitled “METHODS TO ENHANCE MEMORY BY MODULATING 5HT-2A RECEPTOR EXPRESSION” and filed Feb. 8, 2024.

What is claimed:

1. A method of determining a dose of a therapeutic composition to a patient, the patient wearing a wearable device configured to detect at least one physiological parameter of the patient, the method comprising:
detecting the at least one physiological parameter of the patient;
transmitting data associated with the at least one physiological parameter of the patient to a medical professional; and
determining the dose of the therapeutic composition based on the data associated with the at least one physiological parameter of the patient.

2. The method of claim 1, wherein the wearable device comprises a smart ring or a smart watch.

3. The method of claim 2, wherein the wearable device comprises a smart ring.

4. The method of claim 1, further comprising administering at least one dose of the therapeutic composition to the patient.

5. The method of claim 4, wherein the therapeutic composition comprises an RNA therapy.

6. The method of claim 5, wherein the RNA therapy comprises siRNA.

7. The method of claim 5, wherein the RNA therapy comprises shRNA.

8. The method of claim 5, wherein the RNA therapy comprises cRNA.

9. The method of claim 5, wherein administering the RNA therapy comprises:
administering a dose of a neuron-editing biologic intranasally to the patient;
administering a brain region activator to activate a target brain region of the patient; and
transporting the neuron-editing biologic to active neurons within the activated target brain region using hemodynamics, wherein the neuron-editing biologics normalize brain activity within the target brain region.

10. The method of claim 9, further comprising editing the active neurons using the neuron-editing biologics.

11. The method of claim 9, further comprising concentrating neural activity in the target brain region using the brain region activator.

12. The method of claim 1, further comprising selecting a type of the neuron-editing biologics.

13. The method of claim 1, further comprising selecting an appropriate brain region connectome to activate or deactivate the target brain region.

14. The method of claim 1, further comprising selecting the target brain region to be treated to improve the mental health condition.

15. The method of claim 1, further comprising identify brain regions affected by the mental health condition to be treated as the target brain regions.

16. The method of claim 1, further comprising analyzing the data associated with the at least one physiological parameter of the patient with artificial intelligence and analysis algorithms.

17. The method of claim 16, further comprising generating insights and analysis based on an analysis performed by the artificial intelligence and analysis algorithms.

18. The method of claim 17, further comprising sending the insights and analysis based on the analysis performed by the artificial intelligence and analysis algorithms to the patient on a periodic basis.

19. The method of claim 17, further comprising sending the insights and analysis based on the analysis performed by the artificial intelligence and analysis algorithms to the medical professional on a periodic basis.

20. The method of claim 19, wherein determining the dose of the therapeutic composition based on the data associated with the at least one physiological parameter of the patient comprises determining the dose of the therapeutic composition based on the analysis performed by the artificial intelligence and analysis algorithms and the data associated with the at least one physiological parameter of the patient.
